(19) United States
(12) Patent Application Publication
(10) Pub. No.: US 2021/0052833 A1
(43) Pub. Date: Feb. 25, 2021

(54) MOUTHPIECE OF A NEBULIZER, AND PORTABLE NEBULIZER THEREOF

(71) Applicant: Convexity Scientific Inc., Fairfield, CT (US)

(72) Inventors: Vincent F. Carrubba, Baldwin, NY (US); Ralph Finger, Westport, CT (US)

(21) Appl. No.: 16/999,473

(22) Filed: Aug. 21, 2020

Related U.S. Application Data
(60) Provisional application No. 62/890,305, filed on Aug. 22, 2019.

(51) Int. Cl. A61M 15/00

(52) U.S. Cl. CPC A61M 15/0021; A61M 15/009; A61M 2205/583; A61M 2205/8212; A61M 2205/3561

(57) ABSTRACT

The present disclosure discloses a mouthpiece of a nebulizer, comprising an aerosol inflow port, and an aerosol outflow port; wherein an aerosol passage is formed between the aerosol inflow port and the aerosol outflow port, such that in a normal spraying state, the aerosol enters the aerosol passage from the aerosol inflow port and is discharged from the aerosol outflow port; and at least one temporary liquid reservoir is provided inside the mouthpiece, such that when condensation of the aerosol reversely flows back towards the aerosol inflow port, at least part of the condensation of the aerosol flows back into the temporary liquid reservoir. Besides, the present disclosure further discloses a portable nebulizer adopting the mouthpiece. The present disclosure provides an advantage of causing the nebulizer to produce a better anti-backflow effect.

### CROSS REFERENCE TO RELATED APPLICATION

This application claims priority to and incorporates by reference U.S. Patent Application No. 62/890,305 filed Aug. 22, 2019.

### FIELD

The present disclosure relates to the field of medical equipment, and more particularly relates to a mouthpiece of a nebulizer, and a portable nebulizer.

### BACKGROUND

Nebulizers convert a liquid, usually containing a medication, into an aerosol for inhalation by a user. Nebulizers are commonly used for the treatment of asthma, cystic fibrosis, COPD and other respiratory diseases or disorders.

### SUMMARY

Embodiments provides a mouthpiece of a nebulizer, which produces a better anti-backflow effect.

In an embodiment, a mouthpiece of a nebulizer, comprises an aerosol inflow port and an aerosol outflow port; wherein an aerosol passage is formed between the aerosol inflow port and the aerosol outflow port, such that in a normal spraying state, the aerosol enters the aerosol passage from the aerosol inflow port and is discharged from the aerosol outflow port; at least one temporary liquid reservoir is provided inside the mouthpiece, such that when condensation of the aerosol reversely flows back towards the aerosol inflow port, at least part of the condensation of the aerosol flows back into the temporary liquid reservoir.

An aerosol passage is provided inside the mouthpiece. When a relatively large amount of aerosol is present in the mouthpiece, some small-sized condensation will be condensed into relatively large-sized condensation; when the nebulizer is shaken or moved to another location, the aerosol condensation in the mouthpiece possibly flows back, i.e., it is possible to flow back to the aerosol inflow port, while the aerosol inflow port is fitted to the nebulize unit, such that if the condensation flows back to the aerosol inflow port, it is quite possible to flow onto the nebulize unit, producing a pooling on the nebulize unit. However, the embodiments provide, inside the mouthpiece, a temporary liquid reservoir for reducing or mitigating backflow of the condensation to the aerosol inflow port. With such a design, when the aerosol flows back, at least part of the aerosol will flow back to the temporary liquid reservoir, which may significantly reduce the aerosol flowing back to the aerosol inflow port, thereby reducing the odds of producing a pooling on the nebulize unit.

In an embodiment, the mouthpiece comprises a first end wall and a second end wall, which are oppositely disposed, the aerosol inflow port and the temporary liquid reservoir being both disposed at the first end wall, and the aerosol outflow port being disposed at the second end wall.

In an embodiment, the aerosol inflow port is disposed at a middle position of the first end wall, and the temporary liquid reservoir is provided in two, the two temporary liquid reservoirs being disposed at two different sides of the aerosol inflow port.

In an embodiment, a stopper member is provided on an inner side surface of the mouthpiece, for stopping the condensation from flowing back to the aerosol inflow port.

In an embodiment, the stopper member comprises a diverting groove disposed on the inner side surface of the mouthpiece, one end of the diverting groove extending towards the temporary liquid reservoir; or, the stopper member comprises a diverting convex rib disposed on the inner surface of the mouthpiece, one end of the diverting convex extending towards the temporary liquid reservoir.

In an embodiment, the stopper member encloses an isolated area on the inner surface of the mouthpiece, the aerosol inflow port being disposed in the isolated area, the temporary liquid reservoir being disposed outside of the isolated area.

In an embodiment, the temporary liquid reservoir is provided into two; the two temporary liquid reservoirs are disposed at two different sides of the aerosol inflow port; an ancillary air inlet is provided through the inner side surface of the mouthpiece; the aerosol inflow port is disposed at an end wall of the mouthpiece; the stopper member comprises two diverting grooves or diverting convex ribs extending from two sides of the ancillary air inlet to the end wall, and the aerosol inflow port is disposed between the two diverting grooves or diverting convex ribs.

In an embodiment a portable nebulizer comprises a housing, a nebulize unit provided to the housing, and a mouthpiece movably mounted on the housing, the mouthpiece adopting the mouthpiece according to the technical solution above; and in a normal spraying state, the aerosol inflow port is fitted to the nebulize unit.

In an embodiment, a receiving groove is provided on the housing; when the nebulizer is in a non-spraying state, the mouthpiece is received in the receiving groove; when the nebulizer is in a spraying state, the mouthpiece is opened and has a working angle relative to the housing.

In an embodiment, in the received state, one side of the mouthpiece facing the housing is a bottom wall; the working angle is 90°±10°; an opening of the aerosol inflow port is downwardly through the end wall of the mouthpiece to the bottom wall of the mouthpiece.

In an embodiment, the working angle ranges from 70° to 95° , and an opening of the aerosol inflow port extends from an end wall of the mouthpiece to a bottom wall outer surface of the mouthpiece.

In an embodiment, a method, comprises: determining resonant and anti-resonant frequencies of a piezo disk assembly in a nebulize unit; pulsing a signal to the piezo disk assembly at a higher frequency than a target operating frequency, the target operating frequency based on the resonant and anti-resonant frequencies; and nebulizing liquid with the assembly at the target operating frequency.

The target operating frequency may be higher than the resonant frequency by about 10% of the frequency difference between the resonant and anti-resonant frequencies. The determining may comprise measuring impedance of the piezo disk assembly during frequency sweeping of the piezo disk assembly.

The method may further comprise transmitting usage data of the nebulize unit via Bluetooth to a device paired with the apparatus. The method may further comprise indicating a low battery warning when battery power is sufficient for a single 10-minute atomization session.

In an embodiment, a non-transitory computer-readable medium storing instructions that, when executed by one or more computer processors of a device, cause the device to perform operations comprising the methods herein.

In an embodiment, an apparatus may comprise one or more processors; and one or more computer-readable mediums storing instructions that, when executed by the one or more computer processors, cause the apparatus to perform operations comprising the methods herein.

### BRIEF DESCRIPTION OF THE DRAWINGS

Hereinafter, the present disclosure will be described in further detail with reference to the accompanying drawings:

FIG. 1 is a schematic diagram of a mouthpiece in Embodiment 1 of the present disclosure in a received state;

FIG. 2 is a schematic diagram of the mouthpiece in Embodiment 1 of the present disclosure in a normal spraying state;

FIG. 3 is a side schematic diagram of the mouthpiece in Embodiment 1 of the present disclosure in a normal spraying state;

FIG. 4 is a first structural schematic view of the mouthpiece in Embodiment 1 of the present disclosure;

FIG. 5 is a second structural schematic view of the mouthpiece in Embodiment 1 of the present disclosure;

FIG. 6 is an internal structural schematic view of the mouthpiece in Embodiment 1 of the present disclosure;

FIG. 7 is a backflow schematic view of condensation in Embodiment 1 of the present disclosure;

FIG. 8 is a structural schematic view of a bottom wall inner surface of the mouthpiece in Embodiment 1 of the present disclosure;

FIG. 9 is an internal air flow schematic view of the mouthpiece in Embodiment 1 of the present disclosure;

FIG. 10 is a structural schematic view of a stopper member in Embodiment 2 of the present disclosure; and

FIG. 11 is a schematic view of a shape of an aerosol outflow port of the mouthpiece in Embodiment 3 of the present disclosure.

FIG. 12 is a block diagram of a nebulize unit according to an embodiment.

FIG. 13 is a flowchart illustrating a method of using the nebulize unit according to an embodiment.

FIG. 14 is a block diagram illustrating a representative software architecture, which may be used in conjunction with various hardware architectures herein described.

FIG. 15 is a block diagram illustrating components of a machine, according to some example embodiments, able to read instructions from a machine-readable medium (e.g., a machine-readable storage medium) and perform any one or more of the methodologies discussed herein.

### DETAILED DESCRIPTION OF EMBODIMENTS

Hereinafter, the technical solutions of the embodiments of the present disclosure will be explained and illustrated with reference to the accompanying drawings corresponding to the embodiments of the present disclosure. Other embodiments obtained by those skilled in the art without exercise of inventive work based on the examples in the embodiments all fall within the protection scope of the present disclosure.

In the description below, the orientation or position relationships indicated by the terms “inner,” “outer,” “upper,” “lower,” “left,” and “right,” etc. are intended only for facilitating or simplifying description of the present disclosure, not for indicating or implying that the devices or elements have to possess those specific orientations and have to be configured and operated with those specific orientations; therefore, they should not be understood as limitations to the present disclosure.

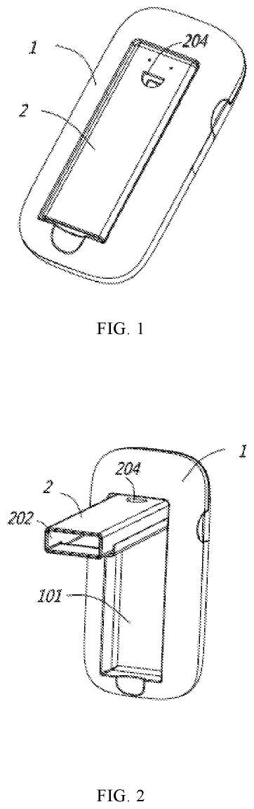

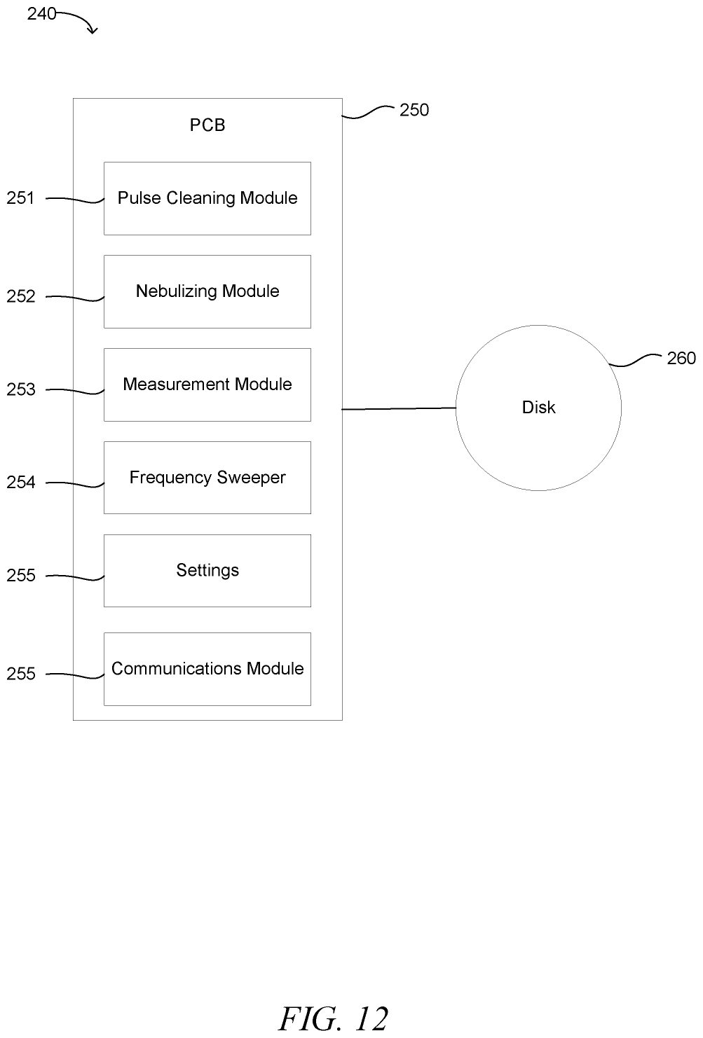

FIGS. 1-9 show an embodiment a mouthpiece 2. FIGS. 1-3 are schematic diagrams of the mouthpiece 2 mounted on a housing 1 of the nebulizer; the mouthpiece 2 according to this embodiment comprises an aerosol inflow port 201 and an aerosol outflow port 202; wherein an aerosol passage is formed between the aerosol inflow port 201 and the aerosol outflow port 202; a main air inlet 203 is further provided on the mouthpiece 2, the main air inlet 203 being in communication with the aerosol passage, such that in a normal spraying state, the aerosol produced by a nebulize unit 240 (FIG. 12) in the nebulizer enters the aerosol passage from the aerosol inflow port 201 and is discharged from the aerosol outflow port 202, wherein a mouth of a user is fitted with the aerosol outflow port 202 such that the aerosol is inhaled into the human body.

An aerosol passage is provided inside the mouthpiece 2. When a relatively large amount of aerosol is present in the mouthpiece, some small-sized condensation will be condensed into relatively large-sized condensation; when the nebulizer is shaken or moved to another location, the aerosol condensation in the mouthpiece 2 likely flows back, i.e., likely flowing back inside the aerosol inflow port 201; further, the aerosol inflow port 201 is fitted to the nebulize unit 240; if the condensation flows back to the aerosol inflow port 201, it very likely flows to the nebulize unit 240, thereby being pooled on the nebulize unit 240; in this embodiment, a temporary liquid reservoir 21 for reducing or mitigating flowback of the condensation to the aerosol inflow port 201 is provided in the mouthpiece 2; with this design, when flowback of the aerosol occurs, at least part of the aerosol will flow back into the temporary liquid reservoir 21, thereby significantly reducing the aerosol flowing back to the aerosol inflow port 201 and further reducing the odds of being pooled on the nebulize unit 240.

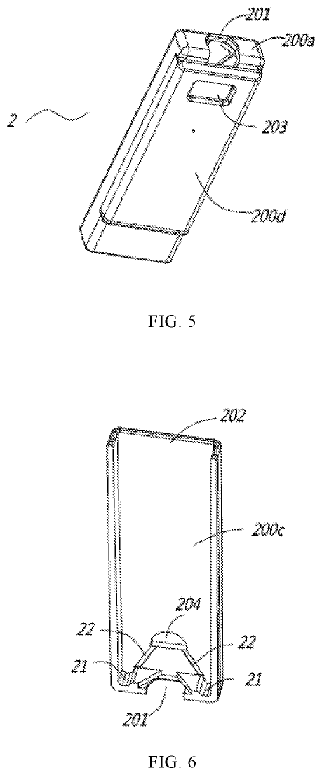

As shown in FIGS. 4 and 5, the mouthpiece 2 in this embodiment is substantially in an elongated cubic shape. The mouthpiece 2 comprises a first end wall 200a and a second end wall 200b which are oppositely arranged; the aerosol inflow port 201 is disposed at the first end wall 200a; the aerosol outflow port 202 is disposed at the second end wall 200b; as further shown in FIGS. 6 and 7, the temporary liquid reservoir 21 and the aerosol inflow port 201 are both disposed at the first end wall 200a, such that the structure of the temporary liquid reservoir 21 may not disrupt the air flow direction inside the mouthpiece 2; if they are disposed on the other inner surface, there may have a hidden risk of disrupting the air flow. Of course, if disruption of the air flow is ignored, the temporary liquid reservoir 21 may be provided on other inner side surface in other embodiments.

Additionally, to better divert the pooled liquid, the aerosol inflow port 201 is disposed at a middle position of the first end wall 200a. The temporary liquid reservoir 21 is provided in two. The two temporary liquid reservoirs 21 are disposed at two different sides of the aerosol inflow port 201, such that when the condensation flows back, it may be diverted to the two sides more uniformly, instead of collectively flowing back into one temporary liquid reservoir 21 thereof, thereby providing a better diverting effect.

Additionally, to further enhance the condensation anti-flowback effect, a stopper member 22 for stopping the condensation from flowing back to the aerosol inflow port 201 is provided on an inner side surface of the mouthpiece 2. By providing the stopper member 22, when the condensation flows back towards the aerosol inflow port 201 from the aerosol outflow port 202, the condensation will be stopped by the stopper member 22, thereby stopping or delaying the condensation from flowing towards the aerosol inflow port 201. The structure and shape of the stopper member 22 are provided in varieties, and this embodiment selects one therefrom, as shown in FIGS. 6 and 7.

In this embodiment, the stopper member 22 comprises a diverting groove disposed on the inner side surface of the mouthpiece 2; one end of the diverting groove extends towards the temporary liquid reservoir 21; the diverting groove plays a role of partitioning the smooth inner surface of the mouthpiece 2, such that when the condensation flows back to the diverting groove, if the amount is small, the condensation is directed to the temporary liquid reservoir 21 along an edge of the diverting groove; if the amount is relatively large, the condensation directly enters inside the diverting groove and then is directed to the temporary liquid reservoir 21.

To further prevent the condensation from flowing back to the nebulize unit 240, this embodiment further improves the aerosol inflow port 201; in a normal spraying state, a bottom wall 200d is provided at one side of the mouthpiece 2 towards the housing 1, and an opening of the aerosol inflow port is through from an end wall of the mouthpiece 2 to an outer surface of the bottom wall 200d of the mouthpiece 2. Such a design has a purpose that when the condensation flows back to the aerosol inflow port 201, part of the condensation directly flows out downwardly from the bottom wall 200d instead of flowing onto the nebulize unit 240, such that less pooling is produced on the nebulize unit 240.

Additionally, in this embodiment, besides providing a main air inlet 203 on the mouthpiece 2, an ancillary air inlet 204 is further provided through an inner side surface of the mouthpiece 2. By providing the ancillary air inlet 204, an air inlet amount increases; further, by providing the ancillary air inlet 204 through the inner side surface, a partition is formed on the inner side surface, which may further play a role of stopping the back-flowing condensation. In this embodiment, as mentioned above, the stopper member 22 comprises two diverting grooves. The two diverting grooves extend to the temporary liquid reservoir 21 from two sides of the ancillary air inlet 204, respectively; in this embodiment, the ancillary air inlet 204 and the two diverting grooves form an isolated area, and the aerosol inflow port 201 is just disposed in the isolated area, while the temporary liquid reservoir 21 is disposed outside of the isolated area. In this way, when the condensation flows back to the first end wall 200a, it is substantially stopped outside the isolated area and substantially does not enter the isolated area.

Moreover, it needs to be noted that the main air inlet 203 of the mouthpiece 2 may be disposed at the bottom wall 200d of the mouthpiece 2; because the main air inlet 203 is provided through the bottom wall 200d inner surface of the mouthpiece 2 and the main air inlet 203 in this embodiment is disposed at a relatively middle position; when the condensation flows back, the main air inlet 203 may also stop the condensation from the bottom wall 200d of the mouthpiece 2 and direct the condensation to flow towards the temporary liquid reservoirs 21 at two sides.

Additionally, this embodiment also improves a forward flow direction of condensation in the mouthpiece 2. The forward flow direction here refers to a direction from the aerosol inflow port 201 towards the aerosol outflow port 202. An aerosol passage is provided inside the mouthpiece 2. When a relatively large amount of aerosol is present in the mouthpiece 2, some small-sized condensation will be condensed into relatively large-sized condensation; the mouthpiece 2 tends to tilt towards the user from time to time when in normal use, such that the condensation also tends to flow towards the aerosol outflow port 202; however, because the aerosol outflow port 202 is directly fitted with the mouth of the user, it is very likely that the condensation directly flows into the user's mouth, which mitigates the efficacy and causes a poor user experience.

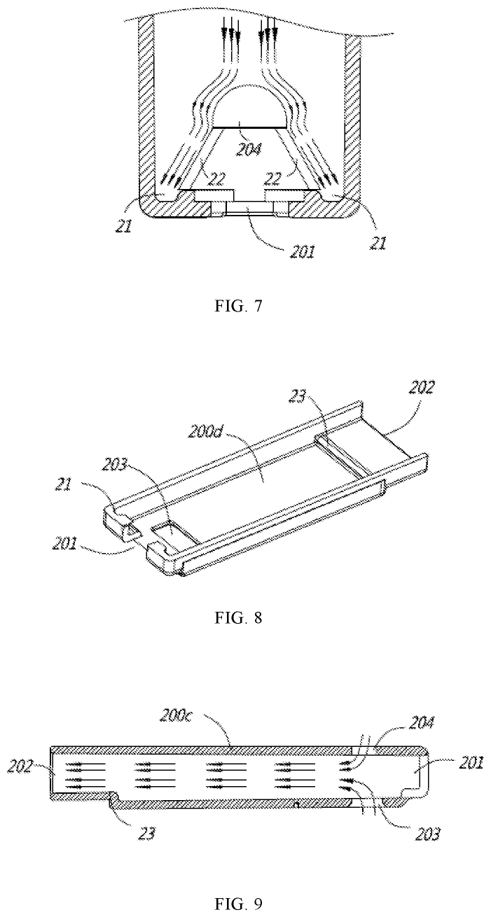

Therefore, to solve this technical problem, an outflow stopper 23 is provided on a bottom wall 200d inner surface of the mouthpiece 2; it needs to be noted that the top wall 200c and the bottom wall of the mouthpiece 2 are described when the mouthpiece 2 is in a normal spraying state. The condensation of the aerosol is stopped by the outflow stopper 23 when flowing towards the aerosol outflow port 202. By providing the outflow stopper 23, most of the condensation remains inside the mouthpiece 2 instead of directly flowing into the user's mouth, specifically referring to FIGS. 8 and 9.

Specifically, the outflow stopper 23 in this embodiment comprises an outflow stopping convex rib protruding from a bottom wall 200d inner surface of the mouthpiece 2; the protruded outflow stopping convex rib may have a better flow stopping effect. In an embodiment, the protrusion height of the outflow stopping convex rib ranges from 1 mm-6 mm. If the protrusion height is too low, the flow stopping effect will be poor; if the protrusion height is too high, an air flow inside the mouthpiece 2 will be disturbed. Therefore, the protrusion height is generally selected to be 3 mm or 4 mm.

It needs to be noted that the outflow stopper 23 in this embodiment may also be an outflow stopping groove provided on a bottom wall 200d inner surface of the mouthpiece 2, a structure of which is similar to the diverting groove above; a role played thereby is to partition a smooth inner surface of the mouthpiece 2 such that a flow stopping effect may be implemented to a certain extent; when the outflow stopper 23 is an outflow stopping groove, a depth of the outflow stopping groove ranges from 1 mm-5 mm; if the depth is too shallow, the flow stopping effect is poor; if the depth is too deep, it will be demanding on the wall thickness of the mouthpiece 2; therefore, the depth of the outflow stopping groove may be 2 mm or 3 mm.

In this embodiment, the shape of the aerosol outflow port 202 of the mouthpiece 2 is matched to the shape of the aerosol outflow port 202, i.e., a rectangular shape. However, considering that some children have smaller mouths, such a rectangular aerosol outflow port 202 provides a poor comfort. Therefore, the shape of the aerosol outflow port 202 may be appropriately adapted, e.g., set to be oval shown in FIG. 11.

Additionally, as mentioned above, in order to increase the air inlet amount, an ancillary air inlet 204 is provided through the inner side surface of the mouthpiece 2 in this embodiment. More specifically, the main air inlet 203 may be disposed at the bottom wall 200d of the mouthpiece 2, while the ancillary air inlet 204 may be disposed at a top wall 200c of the mouthpiece 2. A purpose of providing the ancillary air inlet 204 on the top wall 200c of the mouthpiece 2 is to increase the air inlet amount, which facilitates pushing forward the air flow; meanwhile, because the air inlets are disposed at the top wall 200c and the bottom ball 200d of the mouthpiece 2, the air flow in the mouthpiece 2 is more concentrated in an area between the inner surface of the top wall 200c and the inner surface of the bottom wall 200d, such that the aerosol should be kept away from the top wall 200c and the bottom wall 200d as much as possible, and more aerosol may reach into the user's mouth, instead of being adsorbed to the inner surface of the mouthpiece 2. For details, please refer to the air flow schematic diagram of FIG. 9.

An initial purpose of providing the ancillary air inlet 204 on the top wall 200c of the mouthpiece 2 is to increase the air inlet amount; however, many experiments show that an unexpected effect may be caused by providing the ancillary air inlet 204 there. The diameter of gaseous particles of existing aerosol is substantially between 1 micron and 5 microns; while an actual effective gaseous particle diameter is about 3 microns, because when the gaseous particles have a diameter of about 1 micron, the too small diameter causes the gaseous particles to be exhaled easily when breathing, such that they cannot enter into the body; when the gaseous particles have a diameter of about 4 microns, they generally can only reach the throat; while when the gaseous particles have a diameter of about 5 microns, the larger diameter causes these gaseous particles to substantially only reside in the mouth and unable to be inhaled into the lung. In actual tests, it is found that the gaseous particles with a diameter of about 3 microns is most appropriate and effective, which may be inhaled into the lungs but can be hardly exhaled during breathing.

Further, in this embodiment, after the ancillary air inlet 204 is disposed at the top wall 200c of the mouthpiece 2, many tests find that most of the gaseous particles of the aerosol discharged from the aerosol outflow port 202 have a diameter of about 3 microns. Such kind of aerosol is easily inhaled into the lung, which may significantly enhance the treatment efficacy of the nebulizer.

Additionally, many tests show that the ancillary air inlet 204 may be disposed at a side adjacent to the aerosol inflow port 201. Generally, a maximum distance D1 between the ancillary air inlet 204 and the first end wall 200a does not exceed 4.5 cm; when the distance exceeds 4.5 cm, the gaseous particles with a diameter of around 3 microns among the gaseous particles produced at the nebulizer solution outlet 202 decrease significantly.

Meanwhile, a minimum distance D2 between the ancillary air inlet 204 and the first end wall 200a is no less than 1.2 cm. If the minimum distance is too short, the ancillary air inlet 204 of the mouthpiece 2 top wall 200c will be blocked by the housing 1 in a normal spraying state, such that it cannot play a function of assisting air inlet.

Additionally, the ancillary air inlet 204 in this embodiment has a D shape. Tests show that with the ancillary air inlet 204 of this shape, the air flow inside the mouthpiece 2 is more stable, and more gaseous particles with a diameter of around 3 microns will be produced. Of course, in other embodiments, the ancillary air inlet may be of a rectangular, oval or triangular shape.

### Embodiment II

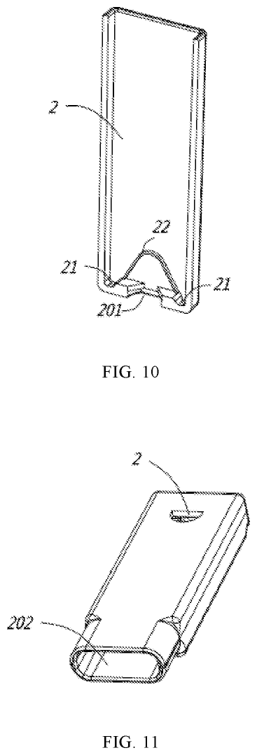

As shown in FIG. 10, the stopper member 22 in this embodiment is not a diverting groove, but a diverting convex rib. When the condensation flows back reversely, it is blocked by the diverting convex rib, thereby implementing an effect of stopping the condensation from flowing back to the aerosol inflow port 201.

Additionally, in this embodiment, an ancillary air inlet 204 is not disposed on the mouthpiece 2; in this embodiment, the entire diverting convex rib encloses an isolated area. In other words, for the isolated area, isolation may be implemented only with the stopper member 22. For example, in this embodiment, the ancillary air inlet 204 and the stopper member 22 jointly form an isolated area, as presented in Embodiment 1.

### Embodiment III

As shown in FIGS. 1-3, a portable nebulizer is provided in this embodiment. The portable nebulizer comprises a housing 1, a nebulize unit 240 provided to the housing 1, and a mouthpiece 2 movably mounted on the housing 1; in this embodiment, the mouthpiece 2 is detachably mounted to the housing 1; for example, they may be fitted via magnetic attachment. The mouthpiece 2 in this embodiment adopts the mouthpiece 2 shown in Embodiment 1 or Embodiment 2 or an equivalence thereto; in the normal spraying state, the aerosol inflow port 201 is fitted to the nebulize unit 240.

The nebulizer in this embodiment is portable such that it is very easy to carry. A receiving groove 101 is provided on the housing 1, such that when the nebulizer is in a non-spraying state, the mouthpiece 2 is received in the receiving groove 101, and when the nebulizer is in a spraying state, the mouthpiece 2 is opened and has a working angle α relative to the housing 1. In other words, in the non-spraying state, the mouthpiece 2 may be received, such that the size of the entire nebulizer does not increase, while in use, the mouthpiece 2 is opened to operate, such that it is very convenient to use.

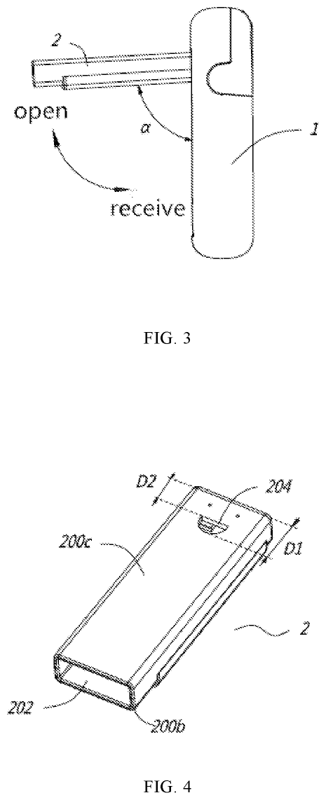

Besides, it needs to be noted that the use angle of the nebulizer in this embodiment is relatively free. Specifically, in the received state, the working angle α ranges from 70° to 95°, e.g., 85°. In such a working angle α, it is user-friendly. The details are shown in FIG. 3.

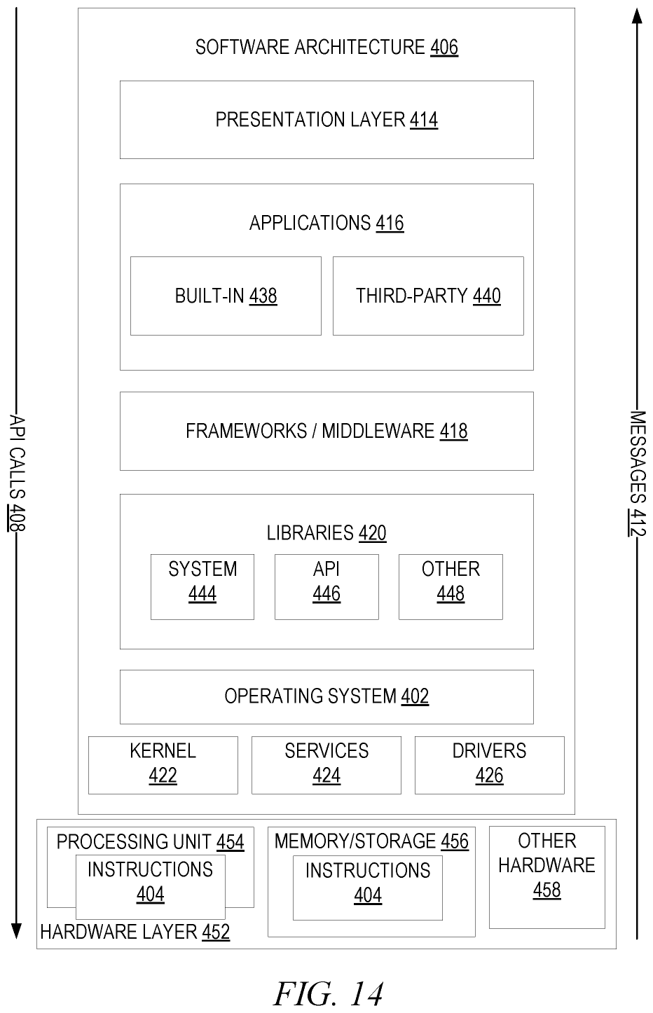

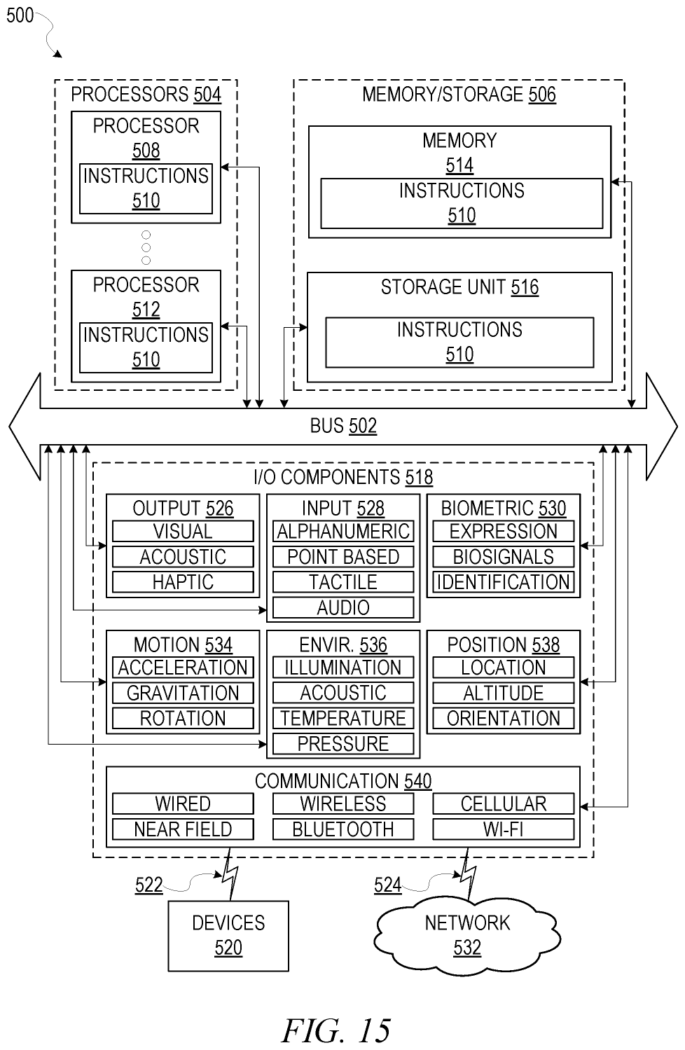

FIG. 12 is a block diagram of a nebulize unit 240 according to an embodiment. The nebulize unit 240 comprises a printed circuit board (PCB) 250 communicatively coupled to a piezo disk assembly (PDA) 260. Other components, such as a power source, are omitted for purposes of clarity. The PCB 250 can be implemented using software architecture 406 (FIG. 14) and machine 500 (FIG. 15). The PCB 250 includes a pulse cleaning module 251, a nebulizing module 252, a measurement module 253, a frequency sweeper 254, settings 255, and communication module 256. The frequency sweeper 254 uses a frequency sweeping approach to determine the resonant frequency of the PDA 260. Upon turning the nebulize unit 240 on, the pulse cleaning module 251 first sends a pulsing signal at 20% higher frequency than a target operating frequency as indicated in the settings 255 to the PDA 260 for 1 to 3 seconds to clean the PDA 260 and help assure best and consistent performance.

Then the nebulizing module 252 sends the target operating frequency and power to the PDA 260 as indicated in the settings 255. The target operating frequency is determined by establishing the resonant and anti-resonant frequencies of the PDA 260. The target operating frequency is higher than the resonant frequency by the value of about 10% of the frequency difference between the resonant and anti-resonant frequencies.

Setting the proper frequency to the PDA 260 can be different from the resonant frequency of the PDA 260 because it tends to damage the PDA 260 and shorten life. The resonant frequency also consumes the most power. This approach which can set the frequency accurately just above the resonant frequency: increases battery lifetime and operational lifetime of the PDA 260 and causes the PDA 260 to use less power.

The measurement module 253 determines the resonant and anti-resonant frequencies by measuring the impedance of the PDA 260 as frequency sweeper 254 sweeps different frequencies on to the PDA 260. The measurement module 253 measures the voltage and current as different frequencies are applied. The PDA 260 is considered equivalent to an RLC circuit which has impedance as the measurable characteristic.

Minimum impedance is the maximum power level used by the PDA and occurs when the PDA is subjected to its resonant frequency. Maximum impedance is the minimum power level used by the PDA and occurs when the PDA is subjected to its anti-resonant frequency. These values can all be stored in the settings 255 for later use and/or be updated regularly at each power on as the target operating frequency can change over time.

The communication module 256 sends information about the nebulize unit 240 activity via Bluetooth or other communication protocol to other devices that, in an embodiment, are paired with it. The information may include date, time, on & off times, etc. of nebulizer usage time. The date and time can be reset or updated whenever the nebulizer pairs with another device.

In addition, the communication module 256 can indicate a battery is low on power even though it has at least one 10 minutes of atomization left. This way the user has a full treatment even if they didn't realize the unit was low on battery. This provides a user the chance to charge the nebulizer after the user receives the last full treatment out of it. The nebulizer can provide approximately 15-20 atomizations. Accordingly, when the battery has approximately 15% of its capacity left . . . that is approximately at least 1 atomization, the communication module with indicate a low battery, e.g., via LED light.

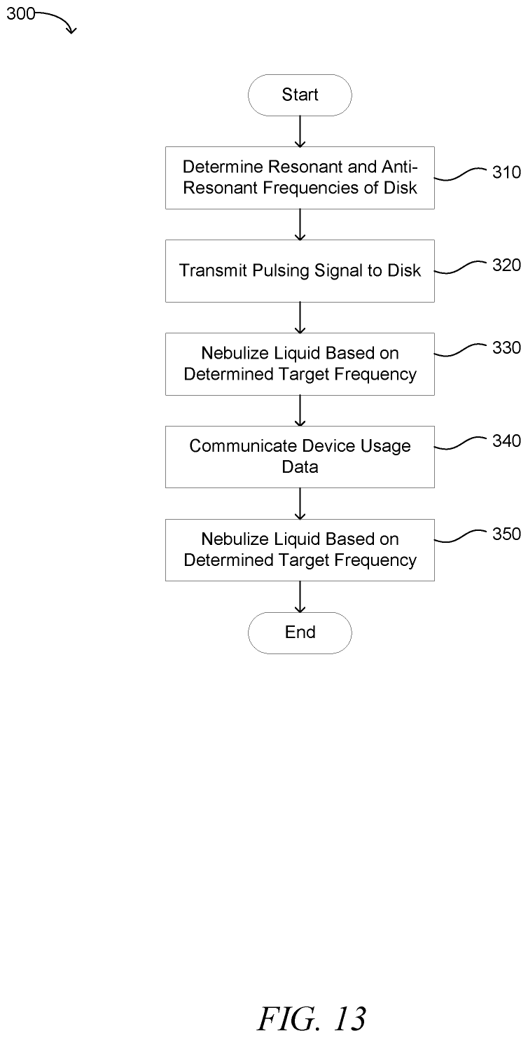

FIG. 13 is a flowchart illustrating a method 300 of using the nebulize unit 240 according to an embodiment. The method determines 310 the resonant and anti-resonant frequencies of the PDA as described above. Next, a pulsing signal is transmitted (320) to the PDA at a frequency higher than the resonant frequency (e.g., by the value of about 10% of the frequency difference between the resonant and anti-resonant frequency). Then liquid is nebulized (330) on the target operating frequency detailed above. The method 300 can further comprise implementing communicating (340) data as discussed above and/or providing (350) a low battery warning as discussed above. The method 300 then ends.

Software Architecture

FIG. 14 is a block diagram illustrating an example software architecture 406, which may be used in conjunction with various hardware architectures herein described. FIG. 14 is a non-limiting example of a software architecture 406 and it will be appreciated that many other architectures may be implemented to facilitate the functionality described herein. The software architecture 406 may execute on hardware such as machine 500 of FIG. 15 that includes, among other things, processors 504, memory 514, and (input/output) I/O components 518. A representative hardware layer 452 is illustrated and can represent, for example, the machine 500 of FIG. 15. The representative hardware layer 452 includes a processing unit 454 having associated executable instructions 404. Executable instructions 404 represent the executable instructions of the software architecture 406, including implementation of the methods, components, and so forth described herein. The hardware layer 452 also includes memory and/or storage modules memory/storage 456, which also have executable instructions 404. The hardware layer 452 may also comprise other hardware 458.

In the example architecture of FIG. 14, the software architecture 406 may be conceptualized as a stack of layers where each layer provides particular functionality. For example, the software architecture 406 may include layers such as an operating system 402, libraries 420, frameworks/middleware 418, applications 416, and a presentation layer 414. Operationally, the applications 416 and/or other components within the layers may invoke API calls 408 through the software stack and receive a response such as messages 412 in response to the API calls 408. The layers illustrated are representative in nature and not all software architectures have all layers. For example, some mobile or special purpose operating systems may not provide a frameworks/middleware 418, while others may provide such a layer. Other software architectures may include additional or different layers.

The operating system 402 may manage hardware resources and provide common services. The operating system 402 may include, for example, a kernel 422, services 424, and drivers 426. The kernel 422 may act as an abstraction layer between the hardware and the other software layers. For example, the kernel 422 may be responsible for memory management, processor management (e.g., scheduling), component management, networking, security settings, and so on. The services 424 may provide other common services for the other software layers. The drivers 426 are responsible for controlling or interfacing with the underlying hardware. For instance, the drivers 426 include display drivers, camera drivers, Bluetooth® drivers, flash memory drivers, serial communication drivers (e.g., Universal Serial Bus (USB) drivers), Wi-Fi® drivers, audio drivers, power management drivers, and so forth, depending on the hardware configuration.

The libraries 420 provide a common infrastructure that is used by the applications 416 and/or other components and/or layers. The libraries 420 provide functionality that allows other software components to perform tasks in an easier fashion than to interface directly with the underlying operating system 402 functionality (e.g., kernel 422, services 424 and/or drivers 426). The libraries 420 may include system libraries 444 (e.g., C standard library) that may provide functions such as memory allocation functions, string manipulation functions, mathematical functions, and the like. In addition, the libraries 420 may include API libraries 446 such as media libraries (e.g., libraries to support presentation and manipulation of various media format such as MPEG4, H.264, MP3, AAC, AMR, JPG, PNG), graphics libraries (e.g., an OpenGL framework that may be used to render 2D and 3D in a graphic content on a display), database libraries (e.g., SQLite that may provide various relational database functions), web libraries (e.g., WebKit that may provide web browsing functionality), and the like. The libraries 420 may also include a wide variety of other libraries 448 to provide many other APIs to the applications 416 and other software components/modules.

The frameworks/middleware 418 (also sometimes referred to as middleware) provide a higher-level common infrastructure that may be used by the applications 416 and/or other software components/modules. For example, the frameworks/middleware 418 may provide various graphic user interface (GUI) functions, high-level resource management, high-level location services, and so forth. The frameworks/middleware 418 may provide a broad spectrum of other APIs that may be used by the applications 416 and/or other software components/modules, some of which may be specific to a particular operating system 402 or platform.

The applications 416 include built-in applications 438 and/or third-party applications 440. Examples of representative built-in applications 438 may include, but are not limited to, a contacts application, a browser application, a book reader application, a location application, a media application, a messaging application, and/or a game application. Third-party applications 440 may include an application developed using the ANDROID™ or IOS™ software development kit (SDK) by an entity other than the vendor of the particular platform, and may be mobile software running on a mobile operating system such as IOS™, ANDROID™ WINDOWS® Phone, or other mobile operating systems. The third-party applications 440 may invoke the API calls 408 provided by the mobile operating system (such as operating system 402) to facilitate functionality described herein.

The applications 416 may use built in operating system functions (e.g., kernel 422, services 424 and/or drivers 426), libraries 420, and frameworks/middleware 418 to create user interfaces to interact with users of the system. Alternatively, or additionally, in some systems, interactions with a user may occur through a presentation layer, such as presentation layer 414. In these systems, the application/component “logic” can be separated from the aspects of the application/component that interact with a user.

FIG. 15 is a block diagram illustrating components of a machine 500, according to some example embodiments, able to read instructions 404 from a machine-readable medium (e.g., a machine-readable storage medium) and perform any one or more of the methodologies discussed herein. Specifically, FIG. 15 shows a diagrammatic representation of the machine 500 in the example form of a computer system, within which instructions 510 (e.g., software, a program, an application, an applet, an app, or other executable code) for causing the machine 500 to perform any one or more of the methodologies discussed herein may be executed. As such, the instructions 510 may be used to implement modules or components described herein. The instructions 510 transform the general, non-programmed machine 500 into a particular machine 500 programmed to carry out the described and illustrated functions in the manner described. In alternative embodiments, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine 500 capable of executing the instructions 510, sequentially or otherwise, that specify actions to be taken by machine 500. Further, while only a single machine 500 is illustrated, the term “machine” shall also be taken to include a collection of machines that individually or jointly execute the instructions 510 to perform any one or more of the methodologies discussed herein.

The machine 500 may include processors 504, memory/storage 506, and I/O components 518, which may be configured to communicate with each other such as via a bus 502. The memory/storage 506 may include a memory 514, such as a main memory, or other memory storage, and a storage unit 516, both accessible to the processors 504 such as via the bus 502. The storage unit 516 and memory 514 store the instructions 510 embodying any one or more of the methodologies or functions described herein. The instructions 510 may also reside, completely or partially, within the memory 514, within the storage unit 516, within at least one of the processors 504 (e.g., within the processor's cache memory), or any suitable combination thereof, during execution thereof by the machine 500. Accordingly, the memory 514, the storage unit 516, and the memory of processors 504 are examples of machine-readable media.

The I/O components 518 may include a wide variety of components to receive input, provide output, produce output, transmit information, exchange information, capture measurements, and so on. The specific I/O components 518 that are included in a particular machine 500 will depend on the type of machine. For example, portable machines such as mobile phones will likely include a touch input device or other such input mechanisms, while a headless server machine will likely not include such a touch input device. It will be appreciated that the I/O components 518 may include many other components that are not shown in FIG. 15. The I/O components 518 are grouped according to functionality merely for simplifying the following discussion and the grouping is in no way limiting. In various example embodiments, the I/O components 518 may include output components 526 and input components 528. The output components 526 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 528 may include alphanumeric input components (e.g., a keyboard, a touch screen configured to receive alphanumeric input, a photo-optical keyboard, or other alphanumeric input components), point based input components (e.g., a mouse, a touchpad, a trackball, a joystick, a motion sensor, or other pointing instrument), tactile input components (e.g., a physical button, a touch screen that provides location and/or force of touches or touch gestures, or other tactile input components), audio input components (e.g., a microphone), and the like.

In further example embodiments, the I/O components 518 may include biometric components 530, motion components 534, environmental components 536, or position components 538 among a wide array of other components. For example, the biometric components 530 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram based identification), and the like. The motion components 534 may include acceleration sensor components (e.g., accelerometer), gravitation sensor components, rotation sensor components (e.g., gyroscope), and so forth. The environmental components 536 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detect concentrations of hazardous gases for safety or to measure pollutants in the atmosphere), or other components that may provide indications, measurements, or signals corresponding to a surrounding physical environment. The position components 538 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 518 may include communication components 540 operable to couple the machine 500 to a network 532 or devices 520 via coupling 524 and coupling 522, respectively. For example, the communication components 540 may include a network interface component or other suitable device to interface with the network 532. In further examples, communication components 540 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 520 may be another machine or any of a wide variety of peripheral devices (e.g., a peripheral device coupled via a USB).

Moreover, the communication components 540 may detect identifiers or include components operable to detect identifiers. For example, the communication components 540 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 540, such as, location via Internet Protocol (IP) geo-location, location via Wi-Fi® signal triangulation, location via detecting a NFC beacon signal that may indicate a particular location, and so forth.

Glossary

“CARRIER SIGNAL” in this context refers to any intangible medium that is capable of storing, encoding, or carrying instructions 510 for execution by the machine 500, and includes digital or analog communications signals or other intangible medium to facilitate communication of such instructions 510. Instructions 510 may be transmitted or received over the network 532 using a transmission medium via a network interface device and using any one of a number of well-known transfer protocols.

“COMMUNICATIONS NETWORK” in this context refers to one or more portions of a network 532 that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network 532 or a portion of a network 532 may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

“MACHINE-READABLE MEDIUM” in this context refers to a component, device or other tangible media able to store instructions 510 and data temporarily or permanently and may include, but is not be limited to, random-access memory (RAM), read-only memory (ROM), buffer memory, flash memory, optical media, magnetic media, cache memory, other types of storage (e.g., erasable programmable read-only memory (EEPROM)), and/or any suitable combination thereof. The term “machine-readable medium” should be taken to include a single medium or multiple media (e.g., a centralized or distributed database, or associated caches and servers) able to store instructions 510. The term “machine-readable medium” shall also be taken to include any medium, or combination of multiple media, that is capable of storing instructions 510 (e.g., code) for execution by a machine 500, such that the instructions 510, when executed by one or more processors 504 of the machine 500, cause the machine 500 to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” excludes signals per se.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors 504) may be configured by software (e.g., an application 416 or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor 504 or other programmable processor 504. Once configured by such software, hardware components become specific machines 500 (or specific components of a machine 500) uniquely tailored to perform the configured functions and are no longer general-purpose processors 504. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor 504 configured by software to become a special-purpose processor, the general-purpose processor 504 may be configured as respectively different special-purpose processors (e.g., comprising different hardware components) at different times. Software accordingly configures a particular processor or processors 504, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses 502) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors 504 that are temporarily configured (e.g., by software) or permanently configured to perform the relevant operations. Whether temporarily or permanently configured, such processors 504 may constitute processor-implemented components that operate to perform one or more operations or functions described herein. As used herein, “processor-implemented component” refers to a hardware component implemented using one or more processors 504. Similarly, the methods described herein may be at least partially processor-implemented, with a particular processor or processors 504 being an example of hardware. For example, at least some of the operations of a method may be performed by one or more processors 504 or processor-implemented components. Moreover, the one or more processors 504 may also operate to support performance of the relevant operations in a “cloud computing” environment or as a “software as a service” (SaaS). For example, at least some of the operations may be performed by a group of computers (as examples of machines 500 including processors 504), with these operations being accessible via a network 532 (e.g., the Internet) and via one or more appropriate interfaces (e.g., an API). The performance of certain of the operations may be distributed among the processors 504, not only residing within a single machine 500, but deployed across a number of machines 500. In some example embodiments, the processors 504 or processor-implemented components may be located in a single geographic location (e.g., within a home environment, an office environment, or a server farm). In other example embodiments, the processors 504 or processor-implemented components may be distributed across a number of geographic locations.

“PROCESSOR” in this context refers to any circuit or virtual circuit (a physical circuit emulated by logic executing on an actual processor) that manipulates data values according to control signals (e.g., “commands,” “op codes,” “machine code,” etc.) and which produces corresponding output signals that are applied to operate a machine 500. A processor 504 may be, for example, a central processing unit (CPU), a reduced instruction set computing (RISC) processor, a complex instruction set computing (CISC) processor, a graphics processing unit (GPU), a digital signal processor (DSP), an ASIC, a radio-frequency integrated circuit (RFIC) or any combination thereof. A processor may further be a multi-core processor having two or more independent processors 504 (sometimes referred to as “cores”) that may execute instructions 510 contemporaneously.

The following examples describe various embodiments of methods, machine-readable media, and systems (e.g., machines, devices, or other apparatus) discussed herein.

1. A mouthpiece of a nebulizer, comprising:
an aerosol inflow port, and
an aerosol outflow port;
wherein an aerosol passage is formed between the aerosol inflow port and the aerosol outflow port, such that in a normal spraying state, the aerosol enters the aerosol passage from the aerosol inflow port and is discharged from the aerosol outflow port; and
at least one temporary liquid reservoir is provided inside the mouthpiece, such that when condensation of the aerosol reversely flows back towards the aerosol inflow port, at least part of the condensation of the aerosol flows back into the temporary liquid reservoir.

2. The mouthpiece of a nebulizer according to example 1, wherein the mouthpiece comprises a first end wall and a second end wall, which are oppositely disposed, the aerosol inflow port and the temporary liquid reservoir being both disposed at the first end wall, and the aerosol outflow port being disposed at the second end wall.

3. The mouthpiece of a nebulizer according to one of the preceding examples, wherein the aerosol inflow port is disposed at a middle position of the first end wall, and the temporary liquid reservoir is provided in two, the two temporary liquid reservoirs being disposed at two different sides of the aerosol inflow port.

4. The mouthpiece of a nebulizer according to one of the preceding examples, wherein a stopper member is provided on an inner side surface of the mouthpiece, for stopping the condensation from flowing back to the aerosol inflow port.

5. The mouthpiece of a nebulizer according to one of the preceding examples, wherein the stopper member comprises a diverting groove disposed on the inner side surface of the mouthpiece, one end of the diverting groove extending towards the temporary liquid reservoir; or, the stopper member comprises a diverting convex rib disposed on the inner surface of the mouthpiece, one end of the diverting convex extending towards the temporary liquid reservoir.

6. The mouthpiece of a nebulizer according to one of the preceding examples, wherein the stopper member encloses an isolated area on the inner surface of the mouthpiece, the aerosol inflow port being disposed in the isolated area, the temporary liquid reservoir being disposed outside of the isolated area.

7. The mouthpiece of a nebulizer according to one of the preceding examples, wherein the temporary liquid reservoir is provided into two; the two temporary liquid reservoirs are disposed at two different sides of the aerosol inflow port; an ancillary air inlet is provided through the inner side surface of the mouthpiece; the aerosol inflow port is disposed at an end wall of the mouthpiece; the stopper member comprises two diverting grooves or diverting convex ribs extending from two sides of the ancillary air inlet to the end wall, and the aerosol inflow port is disposed between the two diverting grooves or diverting convex ribs.

8. A nebulizer, comprising:
a housing,
a nebulize unit provided in the housing, and
a mouthpiece movably mounted on the housing, the mouthpiece adopting the mouthpiece according to the technical solution above; wherein in a normal spraying state, the aerosol inflow port is fitted to the nebulize unit.

9. The nebulizer according to one of the preceding examples, wherein a receiving groove is provided on the housing; when the nebulizer is in a non-spraying state, the mouthpiece is received in the receiving groove; when the nebulizer is in a spraying state, the mouthpiece is opened and has a working angle relative to the housing.

10. The nebulizer according to one of the preceding examples, wherein in the received state, one side of the mouthpiece facing the housing is a bottom wall; the working angle ranges from 70° to 95°; and an opening of the aerosol inflow port extends from an end wall of the mouthpiece to a bottom wall outer surface of the mouthpiece.

11. A method, comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

12. The method of one of the preceding examples, wherein target operating frequency is higher than the resonant frequency by about 10% of the frequency difference between the resonant and anti-resonant frequencies.

13. The method of one of the preceding examples, wherein the determining comprises measuring impedance of the piezo disk assembly during frequency sweeping of the piezo disk assembly.

14. A non-transitory computer-readable medium storing instructions that, when executed by one or more computer processors of a device, cause the device to perform operations comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

15. The medium of one of the preceding examples, wherein target operating frequency is higher than the resonant frequency by about 10% of the frequency difference between the resonant and anti-resonant frequencies.

16. The medium of one of the preceding examples, wherein the determining comprises measuring impedance of the piezo disk assembly during frequency sweeping of the piezo disk assembly.

17. An apparatus, comprising:
one or more processors; and
one or more computer-readable mediums storing instructions that, when executed by the one or more computer processors, cause the apparatus to perform operations comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

18. The apparatus of one of the preceding examples, wherein target operating frequency is higher than the resonant frequency by about 10% of the frequency difference between the resonant and anti-resonant frequencies.

19. The apparatus of one of the preceding examples, wherein the determining comprises measuring impedance of the piezo disk assembly during frequency sweeping of the piezo disk assembly.

20. The apparatus of one of the preceding examples, wherein the operations further comprise transmitting usage data of the nebulize unit via Bluetooth to a device paired with the apparatus.

21. The apparatus of one of the preceding examples, wherein the operations further comprise indicating a low battery warning when battery power is sufficient for a single 10-minute atomization session.

What have been described above are only embodiments of the present disclosure; however, the protection scope of the present disclosure is not limited thereto. A person skilled in the art should understand that the present disclosure includes, but not limited to the contents described in the drawings and the embodiments. Any modifications without departing from the functions and structural principles of the present disclosure will be included within the scope of the claims.

I/we claim:

1. A mouthpiece of a nebulizer, comprising:
an aerosol inflow port, and
an aerosol outflow port;
wherein an aerosol passage is formed between the aerosol inflow port and the aerosol outflow port, such that in a normal spraying state, the aerosol enters the aerosol passage from the aerosol inflow port and is discharged from the aerosol outflow port; and
at least one temporary liquid reservoir is provided inside the mouthpiece, such that when condensation of the aerosol reversely flows back towards the aerosol inflow port, at least part of the condensation of the aerosol flows back into the temporary liquid reservoir.

2. The mouthpiece of a nebulizer according to claim 1, wherein the mouthpiece comprises a first end wall and a second end wall, which are oppositely disposed, the aerosol inflow port and the temporary liquid reservoir being both disposed at the first end wall, and the aerosol outflow port being disposed at the second end wall.

3. The mouthpiece of a nebulizer according to claim 2, wherein the aerosol inflow port is disposed at a middle position of the first end wall, and the temporary liquid reservoir is provided in two, the two temporary liquid reservoirs being disposed at two different sides of the aerosol inflow port.

4. The mouthpiece of a nebulizer according to claim 1, wherein a stopper member is provided on an inner side surface of the mouthpiece, for stopping the condensation from flowing back to the aerosol inflow port.

5. The mouthpiece of a nebulizer according to claim 4, wherein the stopper member comprises a diverting groove disposed on the inner side surface of the mouthpiece, one end of the diverting groove extending towards the temporary liquid reservoir; or, the stopper member comprises a diverting convex rib disposed on the inner surface of the mouthpiece, one end of the diverting convex extending towards the temporary liquid reservoir.

6. The mouthpiece of a nebulizer according to claim 4, wherein the stopper member encloses an isolated area on the inner surface of the mouthpiece, the aerosol inflow port being disposed in the isolated area, the temporary liquid reservoir being disposed outside of the isolated area.

7. The mouthpiece of a nebulizer according to claim 5, wherein the temporary liquid reservoir is provided into two; the two temporary liquid reservoirs are disposed at two different sides of the aerosol inflow port; an ancillary air inlet is provided through the inner side surface of the mouthpiece; the aerosol inflow port is disposed at an end wall of the mouthpiece; the stopper member comprises two diverting grooves or diverting convex ribs extending from two sides of the ancillary air inlet to the end wall, and the aerosol inflow port is disposed between the two diverting grooves or diverting convex ribs.

8. A nebulizer, comprising:
a housing,
a nebulize unit provided in the housing, and
a mouthpiece movably mounted on the housing, the mouthpiece adopting the mouthpiece according to the technical solution above; wherein in a normal spraying state, the aerosol inflow port is fitted to the nebulize unit.

9. The nebulizer according to claim 8, wherein a receiving groove is provided on the housing;
when the nebulizer is in a non-spraying state, the mouthpiece is received in the receiving groove;
when the nebulizer is in a spraying state, the mouthpiece is opened and has a working angle relative to the housing.

10. The nebulizer according to claim 9, wherein in the received state, one side of the mouthpiece facing the housing is a bottom wall; the working angle ranges from 70° to 95°; and an opening of the aerosol inflow port extends from an end wall of the mouthpiece to a bottom wall outer surface of the mouthpiece.

11. A method, comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

12. The method of claim 11, wherein target operating frequency is higher than the resonant frequency by about 10% of the frequency difference between the resonant and anti-resonant frequencies.

13. The method of claim 11, wherein the determining comprises measuring impedance of the piezo disk assembly during frequency sweeping of the piezo disk assembly.

14. A non-transitory computer-readable medium storing instructions that, when executed by one or more computer processors of a device, cause the device to perform operations comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

15. The medium of claim 14, wherein target operating frequency is higher than the resonant frequency by about 10% of the frequency difference between the resonant and anti-resonant frequencies.

16. The medium of claim 14, wherein the determining comprises measuring impedance of the piezo disk assembly during frequency sweeping of the piezo disk assembly.

17. An apparatus, comprising:
one or more processors; and
one or more computer-readable mediums storing instructions that, when executed by the one or more computer processors, cause the apparatus to perform operations comprising:
determining resonant and anti-resonant frequencies of a piezo disk assembly in a nebulize unit;
pulsing a signal to the piezo disk assembly at a higher frequency than a target operating frequency, the target operating frequency based on the resonant and anti-resonant frequencies; and
nebulizing liquid with the assembly at the target operating frequency.

18. The apparatus of claim 17, wherein target operating frequency is higher than the resonant frequency by about 10% of the frequency difference between the resonant and anti-resonant frequencies.

19. The apparatus of claim 17, wherein the determining comprises measuring impedance of the piezo disk assembly during frequency sweeping of the piezo disk assembly.

20. The apparatus of claim 17, wherein the operations further comprise transmitting usage data of the nebulize unit via Bluetooth to a device paired with the apparatus.

21. The apparatus of claim 17, wherein the operations further comprise indicating a low battery warning when battery power is sufficient for a single 10-minute atomization session.
